(19) United States
(12) Patent Application Publication
(10) Pub. No.: US 2009/0149869 A1
(43) Pub. Date: Jun. 11, 2009

(54) SYSTEM AND METHOD FOR PROVIDING SUTURES USING SELF ADHESIVE PADS WITH ANCHORS

(76) Inventor: Eduardo Pena Dolhun, San Francisco, CA (US)

Correspondence Address: Eduardo Pena Dolhun, Suite 302, 2100 Webster Street, San Francisco, CA, 94115, US

(21) Appl. No.: 11/951,275

(22) Filed: Dec. 5, 2007

(51) Int. Cl. A61B 17/10; A61B 17/08; B65D 85/00; A61B 17/06

(52) U.S. Cl. 606/139; 606/214; 606/215; 206/339; 206/63.3

(57) ABSTRACT

The present invention relates to a new and improved system and method for providing sutures using self adhesive pads with anchors. Each self adhesive pad has at least one anchor or hook fixed on top of the pad to hold a suture thread in place or could contain a Micro Velcro System. By applying the pads around an open wound and connecting them with a suture thread, the wound could be closed by tightening the suture thread. The wound could be kept closed by securing the position of both ends of the suture thread. In one embodiment, the end of a suture thread may be anchored on the anchor or hook on the self adhesive pad. The system and method provides improvements over the suture techniques using stitches by securing atraumatic closure of the wound in a non-invasive way.

### BACKGROUND

1. Field of Invention

This invention relates to systems and methods for providing sutures using self adhesive pads with anchors.

2. Background of the Invention

In repairing tissue, the medical caregiver is concerned with various issues including 1) stopping bleeding, 2) preventing infection, 3) addressing cosmetic concerns, and 4) controlling pain. Traditional sutures are threadlike material that medical caregivers (e.g., doctors and veterinarians) use to repair injuries by holding skin, internal organs, blood vessels and other tissue together to facilitate healing and wound closure.

Suturing using traditional stitches requires puncturing the skin tissue using surgical needles, which may cause further trauma to the wound area, and there is a threat of needle stick injuries from sutures used for final skin closure. Also, poorly administrated stitches can lead to cosmetically unappealing tissue surfaces due to local tension on the skin. Furthermore, stitching odd shaped wounds or wounds at locations hard to reach, may present difficult challenges for suturing.

For suturing techniques, there is an issue of user-dependency, because traditional suturing mainly performed by doctors and to a lesser extent by physician assistants, requires expertise and not all doctors or assistants are proficient at suturing. Allowing them to have a more forgiving method may improve the closing of wounds and incision.

Tissue adhesives or surgical glues, e.g. topical cyanoacrylate adhesives (“liquid stitches”), may be used in combination with or as a non-invasive alternative to traditional sutures in wound closure. The adhesive remains liquid until exposed to water or water-containing substances/tissue, after which it cures (polymerizes) and forms a flexible film that bonds to the underlying surface. Surgical glue is less painful to apply than traditional sutures if a wound must be closed without anesthetic. Surgical glue may leave less scarring in some cases, and be easier to care for post-operatively. However, there are limitations in using tissue adhesives, which include contraindications to use in certain area (e.g. near the eyes), difficulty in redoing the process when necessary, and the difficulty of applying glue in certain circumstances.

### SUMMARY OF INVENTION

The present invention relates to a new and improved system and method for providing sutures using self adhesive pads with anchors. In certain embodiments, each self adhesive pad has an anchor or hook fixed on top of the pad to hold a suture thread in place. By applying the pads around an open wound and connecting them with a suture thread, the wound could be closed by pulling or tightening the suture thread like a shoestring, thus applying tension on the wound edges through the anchors and/or hooks fixed on the self adhesive pads. Certain embodiments improve over standard suture techniques by securing atraumatic closure of the wound in a non-invasive way.

Another embodiment includes modifiable self-adhesive strips that run in contour to the wound, which may be shaped and cut by the operator. Also, the suture thread may be replaced by a micro Velcro band or strip. By using an anchor or hook with holes to thread the micro Velcro band or strip through, the wound could be closed using the Velcro fastener surface after pulling or tightening the band or strip.

### BRIEF DESCRIPTION OF THE DRAWINGS

FIG. 1 depicts a system for providing sutures using self adhesive anchors according to one embodiment.

FIG. 2 depicts a flow diagram illustrating the processes for providing sutures using self adhesive anchors according to one embodiment.

FIG. 3 depicts the process of drawing edges of skin openings together by tightening the suture thread that is kept in place by anchors fixed on self adhesive pads according to one embodiment.

FIG. 4 depicts an example of applying the method to an odd shaped wound or skin opening according to one embodiment.

FIG. 5 depicts an example of a self adhesive tape or strip with a group of the adhesive pads attached together with a specified distance between two adjacent pads

FIG. 6 depicts an example of a Micro Velcro System in which a micro Velcro band or strip is used instead of the suture thread.

### DETAILED DESCRIPTION OF EMBODIMENTS OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are, references to the same embodiment; and, such references mean at least one of the embodiments.

A system and method providing sutures using self adhesive anchors are described. The system and method provide improvements over the traditional suture techniques by not puncturing the skin with a surgical needle, securing atraumatic closure of the wound.

The system and method use anchors or hooks attached to self adhesive pads applied on the skin surrounding the wound or cut. A suture thread is connected to each anchor or hook in a zigzag fashion so that when the thread is tightened, the opening of the skin would be closed from the tension applied to the skin through the self adhesive pads. The thread is not fixed on each anchor or hook, but rather kept in place by balancing the tension of the thread from tightening it and the skin opening that tends to stay open if no tension is applied.

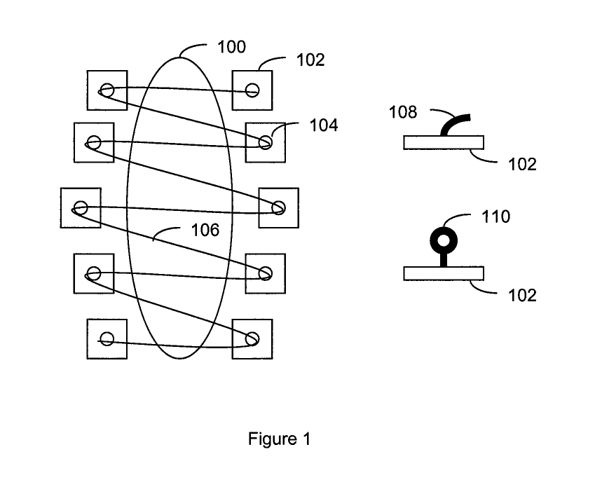

FIG. 1 depicts a system for providing sutures using self adhesive anchors according to one embodiment. The open wound 100 is surrounded by self adhesive pads 102 and each self adhesive pad has a fixed anchor or hook 104. Each pad is attached to the skin at appropriate distances around the wound 100. A suture thread 106 connects each anchor or hook 102. The anchor or hook may be implemented as shown, in the shape of a bent post 108 or a through hole on a post 110.

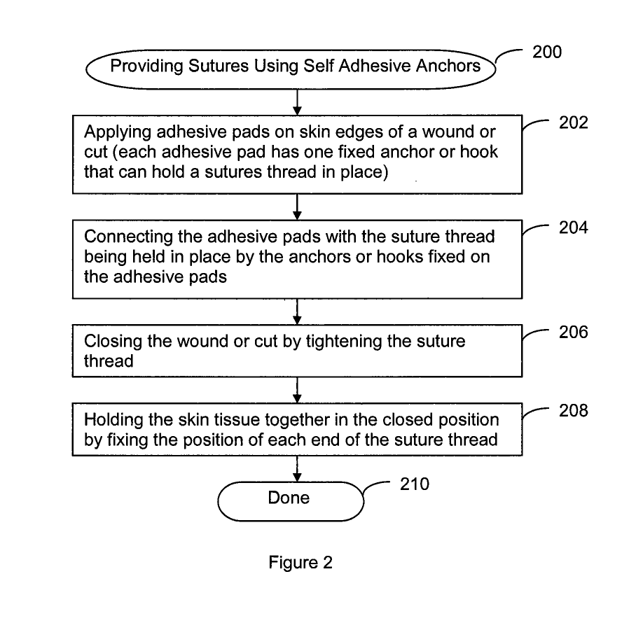

FIG. 2 depicts a flow diagram illustrating the processes for providing sutures using self adhesive anchors 200 according to one embodiment.

In process 202, the methods starts by applying adhesive pads on the skin edges of a wound or cut that needs to be closed. This could be accomplished by attaching each self adhesive pad with an anchor or hook on the skin around the wound one by one. In another embodiment, a self adhesive tape or strip with a group of the adhesive pads attached together with a specified distance between two adjacent pads may be applied on the skin together. In yet another embodiment, a dispenser may be used either to apply one pad at a time or to apply the tape or strip with a group of pads attached to the tape or strip.

In process 204, after the anchors or hooks fixed on the self adhesive pads are in place, a suture thread connects all anchors or hooks 204 to provide tension in order to close the open wound. In process 206, the suture thread is tightened to close the wound or cut. The tension from tightening the thread is applied to the anchors and hooks, which is in turn transferred to the skin to close the wound through the anchors or hooks fixed on the self adhesive pads.

In Process 208, the closed skin is held together in place by fixing the positions of both ends of the thread. The end of thread may be fixed by a variety of ways, e.g. tying the end of the thread to the anchor or hook fixed on the self adhesive pads. In one embodiment, one end of the thread could be fixed to the anchor or hook by having an end with a bigger diameter than the opening of the anchor or hook.

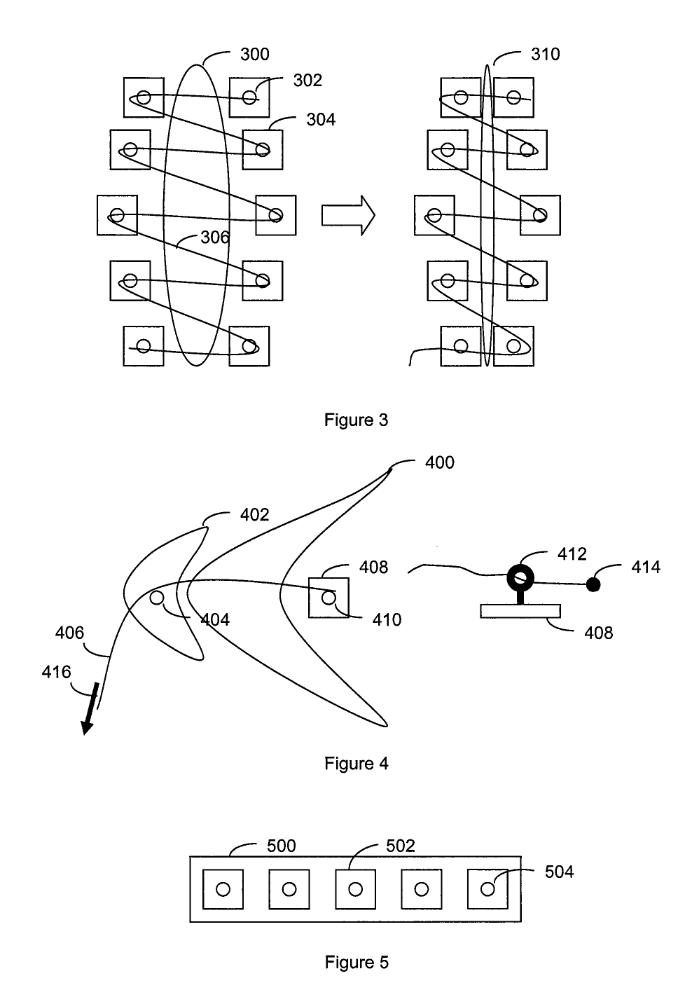

FIG. 3 depicts the process of drawing edges of skin openings together by tightening the suture thread that is kept in place by anchors fixed on self adhesive pads according to one embodiment. Tightening the thread 306 by pulling both ends of the thread applies tension to the skin through the anchors and hooks fixed on the self adhesive pads, and the wound opening can be closed 310.

FIG. 4 depicts an example of applying the method to an odd shaped wound or skin opening 400 according to one embodiment. By having self adhesive pads 402 and 408 with a variety of different shapes, wounds with different shapes may be closed more conveniently. Anchors or hooks 404 and 410 may have different shapes, too. In one embodiment, the anchor 410 has the shape of an opening 412 fixed on a self adhesive pad 408 and one end of the thread 414 has a bigger diameter than the opening 412 of the anchor, thus enabling to apply tension by just pulling the other end of the thread 416.

FIG. 5 depicts an example of a self adhesive tape or strip 500 with a group of the adhesive pads 502 attached together with a specified distance between two adjacent pads. Each pad 502 has an anchor or hook 504 fixed on it. Instead of applying individual pads on the skin, a string of pads could be applied together to the skin by applying the tape or strip 500. In applying the tape or strip 500, a dispenser may be used instead of applying by hand. A dispenser may be also used in applying one pad at a time. Another embodiment of the strip could be a modifiable strip that can be shaped and cut to conform to the wound.

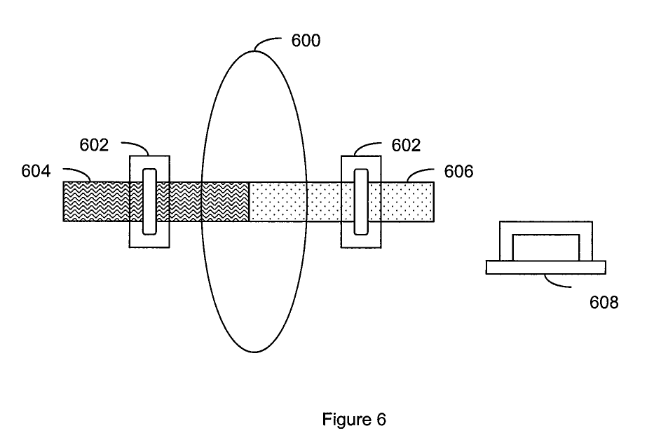

FIG. 6 depicts an example of a Micro Velcro System in which a micro Velcro band or strip is used instead of the suture thread. On the edge of the wound 600, a self adhesive pad having an anchor or hook with a hole in a rectangular shape 602 may be attached. Then the Velcro band or strip 604 606 may be threaded through the anchors or hooks, tightened by pulling the strip, and closed using the Velcro fastener surfaces 604 and 606, just like the pulling or shoestring system. The anchor with a rectangular shape hole 608 is shown to give a side view perspective.

The Velcro surfaces 604 and 606 may be arranged in different locations on the strip and one end of the strip may be fixed to the anchor or hook 602 beforehand.

One embodiment of a suture system may include adhesive pads suitable for applying on skin, anchors or hooks with each one fixed to the adhesive pad, and a suture thread that is held in place by the anchors or hooks. The suture system may have a group of the adhesive pads attached together on a self adhesive tape or strip with a specified distance in between two adjacent pads. Also, the suture system may have the thread having one end that could be anchored on one of the anchors or hooks. The suture system may further include a dispenser that could apply one adhesive pad at a time on skin or a group of pads attached on a self adhesive tape or strip.

One embodiment of a suture method may include processes of applying adhesive pads having anchors or hooks that can hold a suture thread in place on the skin edges of a wound or cut, connecting the adhesive pads with the suture thread being held in place by the anchors or hooks fixed on the adhesive pads, closing the wound or cut by tightening the suture thread, and holding the wound or cut in the closed position by fixing the position of each end of the suture thread. The method may accomplish the process of fixing the position of each end of the suture thread by using a suture thread having one end that could be anchored on the anchor or hook fixed on the adhesive pad by having a bigger diameter than the anchor or hook opening or by tying the thread to the anchor or hook fixed on the adhesive pad.

The suture method may use a group of the adhesive pads that are attached together on a self adhesive tape or strip with a specified distance in between two adjacent pads. Also, the suture method may use a thread having one end that could be anchored on one of the anchors or hooks by having a bigger diameter than the anchor or hook opening. The suture method may apply adhesive pads on skin edges of a wound or cut by using a dispenser that could apply one adhesive pad at a time on skin or a group of pads attached on a self adhesive tape or strip.

An example of self adhesive pads in many different shapes suitable for application on skin may have different anchors or hooks fixed on top of the pad to hold a suture thread in place. The self adhesive pad may be attached to a tape or strip with a length to hold many pads.

For example, modifiable adhesive strips that run in contour to the wound may be shaped and cut by the operator. Also, a micro Velcro band or strip could be used instead of the suture thread (Micro Velcro System). The Micro Velcro System could use a hook with a rectangular shape hole to thread the micro Velcro strip, tightened by pulling the strip, and closed using the Velcro fastener surface, just like the pulling or shoestring system.

Although the above embodiments have been described with reference to specific example embodiments, it will be evident that the various modification and changes can be made to these embodiments. Accordingly, the specification and drawings are to be regarded in an illustrative sense rather than in a restrictive sense. The foregoing specification provides a description with reference to specific exemplary embodiments. It will be evident that various modifications may be made thereto without departing from the broader spirit and scope as set forth in the following claims. The specification and drawings are, accordingly, to be regarded in an illustrative sense rather than a restrictive sense.

What is claimed is:

1. A suture system comprising:
a plurality of adhesive pads suitable for applying on skin;
a plurality of anchors or hooks that are fixed to the adhesive pads; and
a suture thread that is held in place by the anchors or hooks.

2. The suture system of claim 1, wherein a group of the adhesive pads are attached together on a self adhesive tape or strip with a specified distance in between two adjacent pads.

3. The suture system of claim 2, further comprising: a dispenser that could apply the self adhesive tape or strip on skin.

4. The suture system of claim 1, wherein the thread has one end that could be anchored on one of the anchors or hooks.

5. The suture system of claim 1, further comprising
a dispenser that could apply one adhesive pad at a time on skin.

6. The suture system of claim 1, wherein the adhesive pad is a modifiable strip that can be shaped or cut to conform to the wound

7. A suture method comprising:
applying a plurality of adhesive pads on skin edges of a wound or cut, wherein each adhesive pad has at least one fixed anchor or hook that can hold a suture thread or a Velcro strip in place, wherein the Velcro band or strip has a first Velcro fastener surface having a plurality of micro hooks and a second Velcro fastener surface having a plurality of micro loops;
connecting the adhesive pads with the suture thread or a Velcro strip being held in place by the anchors or hooks fixed on the adhesive pads;
closing the wound or cut by tightening the suture thread or a Velcro strip; and
holding the wound or cut in the closed position by fixing the position of each end of the suture thread or by closing the Velcro strip using the Velcro fastener surfaces.

8. The suture method of claim 7, wherein fixing the position of one end of the suture thread is accomplished by using a suture thread having at least one end that could be anchored on the anchor or hook fixed on the adhesive pad by having a bigger diameter than the anchor or hook opening.

9. The suture method of claim 7, wherein fixing the position of each end of the suture thread is accomplished by tying at least one end of the thread to the anchors or hooks fixed on the adhesive pad.

10. The suture method of claim 7, wherein a group of the adhesive pads are attached together on a self adhesive tape or strip with a specified distance in between two adjacent pads.

11. The suture method of claim 7, wherein the thread has one end that could be anchored on one of the anchors or hooks by having a bigger diameter than the anchor or hook opening.

12. The suture method of claim 7, wherein applying a plurality of adhesive pads on skin edges of a wound or cut is done using a dispenser that could apply one adhesive pad at a time on skin.

13. The suture method of claim 10, wherein applying a plurality of adhesive pads on skin edges of a wound or cut is done using a dispenser that could apply the self adhesive tape or strip on skin.

14. A self adhesive pad suitable for application on a skin, with at least one anchor or hook fixed on top to hold a suture thread in place.

15. The self adhesive pad of claim 14, which is attached to a tape or strip with a length to hold a plurality of such self adhesive pads.

16. A suture system comprising:
a plurality of adhesive pads suitable for applying on skin;
a plurality of anchors or hooks that are fixed to the adhesive pads; and
a Velcro band or strip that is held in place by the anchors or hooks and closed using the Velcro band or strip, wherein the Velcro band or strip has a first surface having a plurality of micro hooks and a second surface having a plurality of micro loops.

17. The suture system of claim 16, wherein one end of the strip is fixed to one of the anchors or hooks.
